(19) United States
(12) Patent Application Publication
(10) Pub. No.: US 2005/0234375 A1
(43) Pub. Date: Oct. 20, 2005

(54) Splint or support with quick location technique

(75) Inventors: Tracy E. Grim, Bixby, OK (US); Wendy Henderson, Ventura, CA (US); Joseph M. Iglesias, Newbury Park, CA (US); Michael Campos, Sylmar, CA (US); Walter Doubleday, Jupiter, FL (US); Kelly M. Speakes, Woodland Hills, CA (US)

Correspondence Address: FULWIDER PATTON LEE & UTECHT, LLP;Attention : Alan C. Rose, Howard Hughes Center, Tenth Floor, 6060 Center Drive, Los Angeles, CA, 90045, US

(73) Assignee: Royce Medical Company

(21) Appl. No.: 10/828,744

(22) Filed: Apr. 20, 2004

(51) Int. Cl. A61F 5/00

(52) U.S. Cl. 602/8

(57) ABSTRACT

An orthopaedic splint technique involves the use of a water hardenable blank, with (1) primary tacking arrangements for adjustably holding the splint or support in place after it has been activated and placed on the injured part of the anatomy, and may also include (2) functional arrangements for holding the splint firmly in place following accurate mounting and location of the splint on the injured part of the anatomy. The tacking arrangements may involve the use of lateral extensions from the main body of the splint with hook and loop type arrangements or adhesive associated with the extensions for holding the splint onto the injured part of the anatomy.

### BACKGROUND OF THE INVENTION

This invention relates to orthopaedic splints or supports.

In the field of hardenable orthopaedic splints and supports, the splints are normally rectangular in shape and are held on to the anatomy by a finishing tape of some kind wrapped along the entire length of the splint. To function properly, both the splint and finishing tape are soft and supple in order to conform to the contours of the anatomy. During application of the splint, it is necessary that the limb be held in a variety of positions. Frequently, lengthy splints must be used to properly immobilize the injured limb, and gravity causes one end or the other of the splint to fall away from the anatomy or merely shift away from the proper position. If one person is attempting to apply the splint, it is difficult to maintain the appropriate position for each particular injury during hardening. Usually it is necessary to ask for additional assistance to ensure proper application. More particularly, an extra set of hands is required to keep the splint in the desired location on the patient's anatomy until the outer securing or immobilization means has been applied.

### SUMMARY OF THE INVENTION

In accordance with the present invention a splint or support is provided with primary tacking arrangements to locate the splint in place, allowing the physician or technician the use of two free hands for quickly and easily adjusting the position of the splint blank if necessary without removing or loosening said arrangements. The user then applies secondary more supportive securing arrangements to effectively hold the splint or support to the injured part of the anatomy.

In accordance with one illustrative implementation of the invention, the primary tacking arrangements may involve mating hook and loop type material such as VELCRO® type inserts or pads, or hook type patches used with a splint covering of unbroken loop (UBL) or other hook receivable material on the surface of the splint. In this regard the entire hook receivable surface acts as the loop portion of the hook and loop fastening arrangements.

In addition, with the splint or support normally being longitudinal in extent, laterally extending securing extensions may be provided with hook and loop, adhesive or other securing arrangements at the outer ends thereof. These lateral extensions may be formed as part of the hardenable portion of the splint, or as part of one layer thereof, or may be separate straps or the like, secured to the splint blank.

The secondary or “functional” securing arrangements may be in the form of a stretchable wrap such as an ACE® bandage, or may be straps, where the said arrangements are of sufficient strength to firmly hold the splint to the injured part of the anatomy during regular usage for extended periods of time.

Other features which may be included in implementation of the invention may involve the following:

1. The use of “spacer” type double knit material for the splint.

2. The use of an outer or secondary support such as an exo-skeleton type support, with associated straps serving as the functional securing arrangements.

3. The use of non-rectangular splints, with laterally extending areas forming the primary securing arrangements.

4. The use of a roll of splinting material, with lateral extensions spaced along its length.

5. The use of splint blanks with a thumbhole or another web space locater forming a part of the assembly.

In the field of splinting it is normally desirable to fully secure the splint or cast so that it will not come off or shift location during the normal course of daily activities. The primary holding or splint locating arrangements in the present invention are used when the splint blank is flexible and is being initially applied. These primary securing arrangements may be considered to be temporary “tacking” or locating arrangements, as they permit easy adjustment of the splint position; and are usually of insufficient strength to rigidly immobilize the splint for long term use by the patient.

Accordingly, when the term “tacking” is used in the present specification and claims, reference is being made to the primary holding arrangements which are of insufficient strength to fully secure the splint; and subsequent securing arrangements are normally required in addition to the “tacking”. The “tacking” may be implemented, for examples, by hook and loop fastening arrangements, by adhesive, by snaps, by hooks or any other suitable arrangement; and functional securing of the splint may be accomplished by lamination of the layers when the edges of hardenable material are overlapped, by wrapping with flexible tape, such as an ACE® bandage tape, by straps, or by any other securing arrangement of sufficient strength to properly immobilize the splint to the injured portion of the anatomy.

It is further noted that the use of a layer of UBL or hook-receivable material has the additional advantage of restricting the flow of the hardenable material, as set forth in co-pending U.S. patent application Ser. No. 10/136,023, filed Apr. 29, 2002.

In accordance with a method illustrating certain aspects of the invention, a splint or support is formed with sheet material impregnated with hardenable material, and with one or more primary laterally extending tacking extensions. The hardenable material is activated and the splint or support is held to the anatomy by engaging the laterally extending extensions; and the position and/or configuration of the impregnated sheet material is adjusted prior to the hardening of the hardenable sheet material while it is being loosely held by the tacking extensions; and the adjusted position is maintained while the hardening is accomplished.

Other objects, features and advantages will become apparent from a consideration of the following detailed description and the accompanying drawings.

### BRIEF DESCRIPTION OF THE DRAWINGS

FIG. 1 is perspective view of a short arm splint embodiment illustrating the principles of the invention;

FIG. 2 is a partial cross-sectional view of part of the assembly of FIG. 1 prior to application;

FIG. 3 shows a splint roll with laterally extending tacking arrangements;

FIG. 4 shows a longitudinally extending splint or support blank with tacking arrangements extending from one side thereof;

FIG. 5 illustrates a splint blank with shaped tacking arrangements extending from both sides thereof;

FIG. 6 shows a non-rectangular splint or support blank with laterally extending tacking arrangements;

FIG. 7 is a showing of a splint blank with thumb holes within the laterally extending tacking arrangements;

FIGS. 8 and 9 are perspective and cross-sectional views, respectively, of another embodiment of the invention.

FIG. 10 shows a splint or support, and an outer exo-skeletal construction;

FIG. 11 illustrates a splint or support for the forearm and thumb, with an attachment extending through the web space between the thumb and forefinger; and

FIG. 12 shows a splint for the upper and lower arm with an “anti-flexion” strap.

### DETAILED DESCRIPTION OF THE PREFERRED EMBODIMENTS

While the specification describes particular embodiments of the present invention, those of ordinary skill can devise variations of the present invention without departing from the inventive concept.

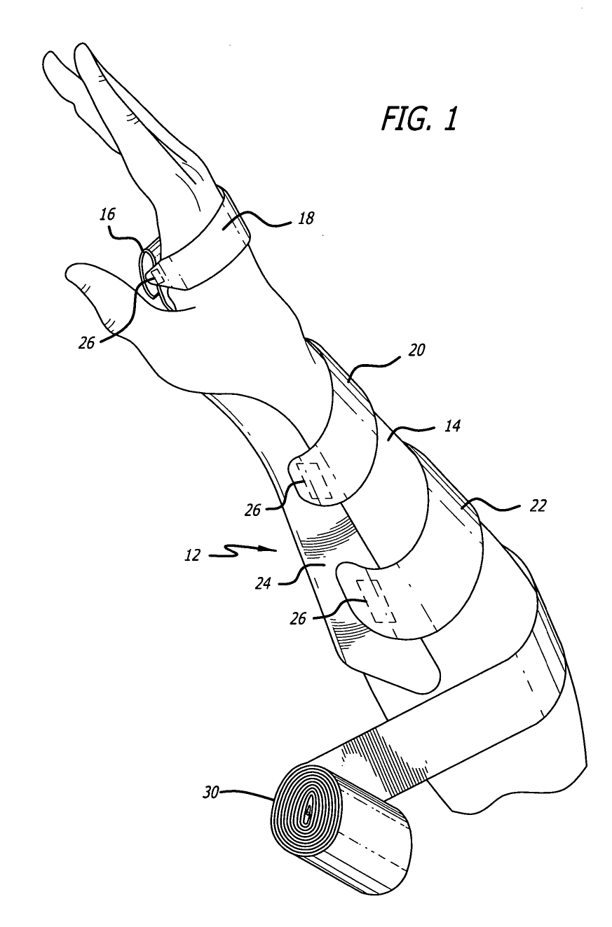

Referring more particularly to the drawings, FIG. 1 shows a splint or brace 12 mounted on the forearm 14 of a patient. The main part of the brace 12 is optionally folded over at reference numeral 16 at the palm of the patient's hand, and strap 18 extends over the back of the hand. The straps 18, 20 and 22 are secured to or are part of the main part 24 of the splint, and are provided with hook type fastener 26 on the underside of straps 18, 20 and 22 as shown in FIG. 1. The areas of the splint 12 underlying patches 26 are of hook receivable type material, so the straps 18, 20 and 22 are held or “tacked” in place as the splint 12 is applied. With the splint being held onto the forearm in an adjustable manner, the physician or medical technician can readily position the splint to the precise desired location, without the assistance of additional medical personnel.

The splint location is subsequently immobilized by wrapping the flexible, resilient tape 30 around the forearm and splint. This tape 30 may be of the type sold under the ACE® trademark.

Incidentally, relative to the hook receivable type material underlying the hook type patches 26, it may either be in the form of hook receivable type patches secured to the splint 12 in selected areas; or alternatively, the entire splint may be covered with a layer of unbroken loop (UBL) or other hook receivable material, so that the hook type patches will engage and secure to the splint 24 at any convenient location.

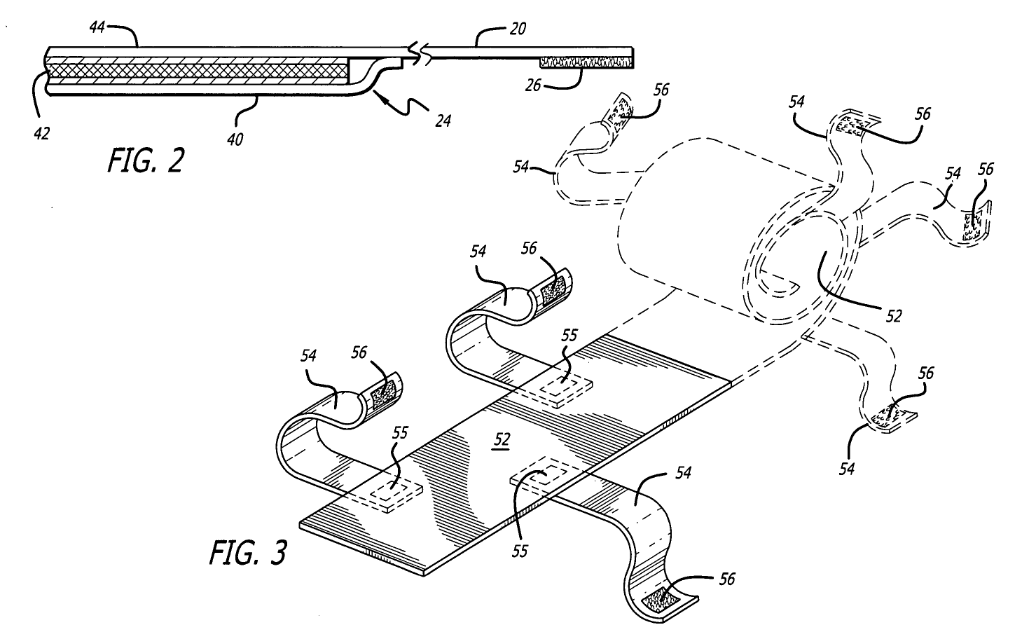

Incidentally, instead of VELCRO® type fasteners, adhesive, snaps, or hooks, for examples, could be employed for tacking the straps 18, 20 and 22 in place. FIG. 2 is a schematic cross-sectional view of a part of the assembly of FIG. 1 prior to application. In FIG. 2, the main portion 24 of the splint 12 includes an inner padding layer 40, and a central layer of hardenable material 42 preferably made of a double knit spacer material. The double knit spacer material includes upper and lower woven or knit layers, with an integral matrix of fibers or filaments interconnecting the upper and lower layers. An additional layer of unbroken loop (UBL) or hook receivable material 44 is adjacent to the upper surface of the spacer material 42. The strap 20 may be formed of separate material, or may be a continuation of any one of the layers of the main body 24 of the splint blank. It may also be either permanently attached or removably attached, using VELCRO®, hooks, etc. At the outer end of strap 20 is a patch 26 of hook type material for securing to underlying hook receivable type material 44.

The hardenable layer 42 is preferably impregnated with a water activatable material such as urethane. The layer of hook receivable or UBL material 44 may inhibit the transfer of the water activatable material to the outer surface of the splint.

FIG. 3 shows a continuous roll of a splinting material 52 with laterally outwardly extending tacking arrangements 54 having patches 56 of hook type material located at the ends thereof. The main body of the splint roll 52 consists of at least one layer of material impregnated with a water activatable material. The outer surface of the splint roll 52 may be covered with hook receivable material or may be provided with patches of hook receivable type material for tacking engagement with the hook type patches 56. The tacking strips 54 may be in the form of separate strips with hook type patches 55 at the inner ends of strips 54 as well as on the outer ends of strips 54 where hook type patches 56 are provided. This gives flexibility in the location of the tacking arrangements of the splint onto the anatomy. Alternatively, the tacking strips 54 may be permanently secured to the splinting material 52 or the strips 54 may be an extension or continuation of one of the layers of splinting material 52. A splint or support may be cut from the roll at any desired length to form a blank of the desired size.

The preferred splint roll configuration is an inner activatable material of at least one layer with an outer covering material on one side and a padding layer on the other. The outer covering is preferably made from a hook receivable material, such as a UBL material, and the padding may consist of at least one layer of either a double knit spacer material, foam, or a nonwoven material. Throughout the length of the splint are tacking arrangements spaced at equal intervals. The extensions may have hook type arrangements on both ends so that they would be removable and movable with respect to the splint assembly. It is also preferable that the tacking arrangements be made of a stretchable material and have a configuration that would ensure proper securing yet not cause any discomfort to the end user. An acceptable material to use would be a nonwoven because of its cost effectiveness and low profile. The splint can be cut to usable lengths prior to packaging or may be provided in long lengths and may be cut to the appropriate size immediately prior to application.

In the case of all of the embodiments as disclosed in this specification, the construction and alternatives as disclosed herein may be used in each of the constructions intended for specific application. Thus for example, after cutting off a length of the splint roll 52, activating it, and tacking it in place, the splint may be wrapped with a flexible wrap, to functionally secure the splint.

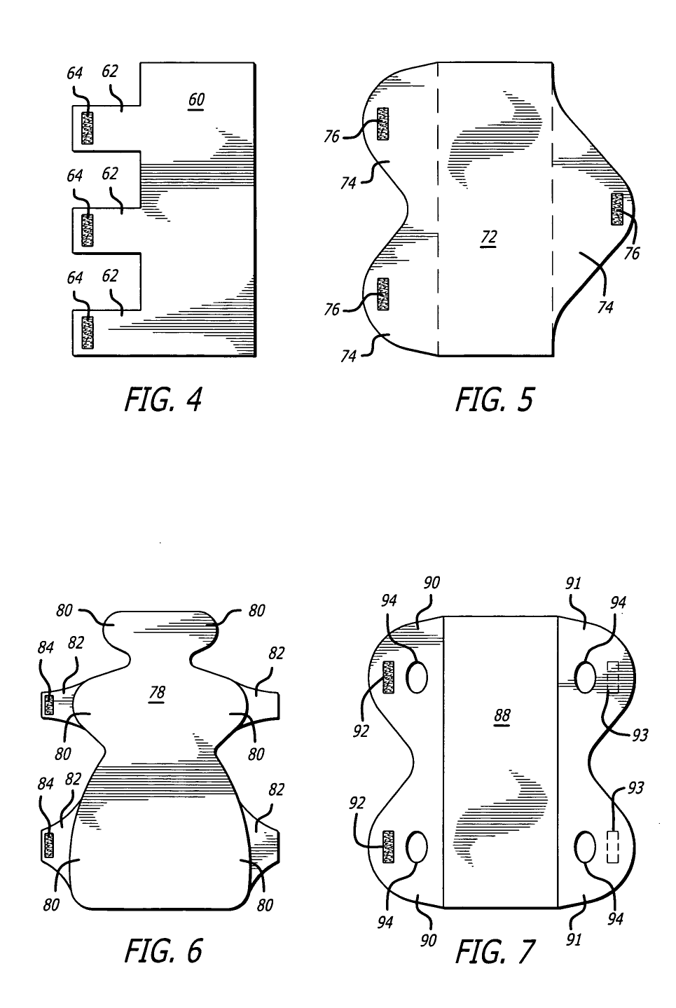

FIGS. 4 through 7 show further embodiments of the invention with different geometric configurations. In FIG. 4 the longitudinally extending main blank 60 has laterally extending tacking arrangements 62 extending from one side. Hook type patches 64 are provided at the outer ends of the tacking extensions 62, to mate with underlying hook receivable type material. In use, these extensions 62 are wrapped around the anatomy and secured to either the outer hook receivable covering of the splint 60 or to strategic areas where hook receivable patches have been placed on the surface of the blank 60. The lateral extensions may be an extension of the covering material, padding material, hardenable material, or may be made from an entirely separate material attached to the splinting assembly. The extensions may also be either permanently attached to the splint, i.e., by sewing, or may be removably attached using arrangements such as a hook type fastener.

In FIG. 5 the main longitudinally extending splint blank 72 has shaped laterally extending tacking arrangements 74 with hook type patches 76. In this particular example, the shaped tacking arrangements are staggered and sufficiently wide to allow for complete coverage of the arm when in use, giving circumferential compression and a broader distribution of the forces exerted by the arrangements than would be provided by a narrower strip.

In FIG. 6 the hardenable portion of the splint blank 78 has a “snowman” like configuration, with rounded lateral zones 80. The rounded or contoured shape of the hardenable blank can be strategically designed to better fit and mold to the complex contours of the anatomy than a rectangular splint with no contours. To these lateral zones 80 are secured laterally outwardly extending tacking extensions 82 with mating hook patches 84 and hook receivable patches (or a UBL layer) underlying these extensions when the splint is applied. Instead of having one set of extensions as in FIG. 5, FIG. 6 shows two sets of extensions which will both wrap partially around the anatomy and mate with each other to provide the tacking arrangements.

The splint blank of FIG. 7 has a longitudinally extending central zone 88 and shaped laterally extending zones 90 and 91. Having shaped extensions instead of rectangular strips provides for wider coverage and more pressure distribution over the strapping area, thus reducing potential pressure points associated with a thinner strip. The laterally extending zones 90 have hook type patches 92 to engage mating hook receivable type material 93 on the paired zones 91 on the other side of the blank 88. Alternatively, the entire surface of zones 90 and 91 may be of a hook receivable material and thus the patches 93 would be unnecessary. In use, the thumb is slid through one of the pre-made thumb holes 94 which provide assistance in locating the blank 88 on the forearm of the patient. Multiple thumb holes are provided so that application is possible, regardless of the initial orientation of the splint when removed from the package. Other methods of creating a thumb hole include providing slits in the zones 90 and 91 or making the laterally extending zones out of a tearable material which can be torn to receive a thumb in the appropriate location immediately prior to application.

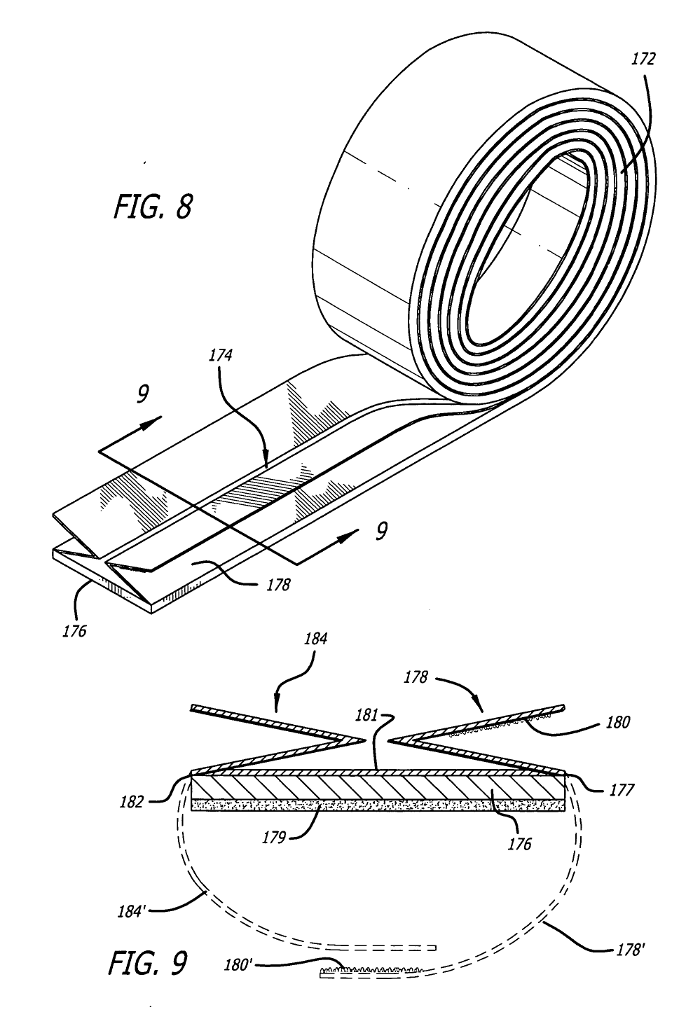

Referring now to FIGS. 8 and 9 of the drawings, FIG. 8 is a perspective view of a water hardenable splint or support in roll form; and FIG. 9 is a cross-sectional view taken along the plane indicated at 9-9 in FIG. 8. In FIG. 8, the roll 172 is partially unrolled to show a section 174 which may be cut off at the desired length.

As shown to advantage in FIG. 9 the splint or support may have a base 176 of spacer material impregnated with water hardenable material. Extending along and secured to one edge 177 of the base 176 is a strip of folded sheet material 178 which has hook material 180 located on one surface thereof as indicated in FIG. 9. From the other edge 182 of the base 176, a second folded strip 184 is secured. The strip 184 may be formed of hook receivable sheet material, such as UBL material or a non-woven fabric.

Shown in dashed lines are the strips 178′ and 184′ in an unfolded configurations, to extend around a portion of the anatomy such as a forearm or lower leg, for examples. The hook material 180′ engages the lower surface of the unfolded hook receivable strip 184′ to hold the base member 176 onto the desired part of the anatomy. This arrangement permits tacking of the base strip 176 in place while the physician or medical technician is adjusting the position of the splint or support.

As shown in FIG. 9, padding material 179 may be provided underlying the strip 176 which is impregnated with hardenable material. With this arrangement, the strip of hardenable material 176 does not directly engage the patient's skin. Incidentally, instead of being formed of spacer type double knit material, the strip 176 may be formed of multiple layers of single knit material. The padding material 179 may be composed of a layer of double kit material. The strip 176 may be covered with a layer of hook receivable material 181 which may have extensions such as those shown at 178 and 184. Thus, the material 181 may be integral with strips 178 and 184; or the strips 184 and 178 may be separately secured to the edges of strip 176. Alternatively, the padding material may extend outwardly to form the securing strips 178 and 184. The foregoing are merely examples of alternative embodiments and many different combinations are possible which incorporate the various configurations discussed above.

The assembly as shown in FIGS. 8 or 9, may subsequently be wrapped with an ACE® type strip material. Alternatively, if the patient is not active, the strips 178′ and 184′ may provide adequate strength or securing force so that additional, secondary securing arrangements are not needed.

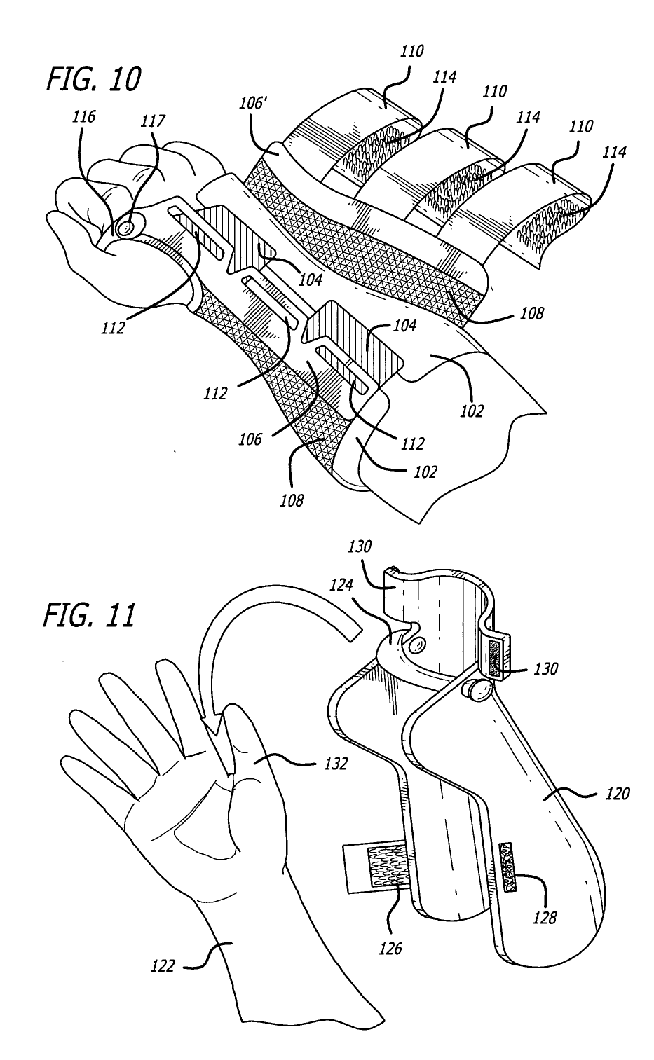

The embodiment of FIG. 10 includes an inner blank 102 with tacking arrangements 104 which engage the underlying opposing edge of inner blank 102. A preshaped outer external wrap can serve as the functional securing arrangements; for example, FIG. 10 is one specific example of these arrangements where exo-skeletal members 106 and 106′ are formed of fairly stiff plastic and are molded to the flexible material 108 which extends around and over the inner splint 102. The plastic strip 106′ is molded to the flexible material 108 at the edge of material 108 away from exo-skeletal member 106. Functional securing straps 110 extend through loops 112 and are then folded back upon themselves and secured in place by mating hook and loop securing surfaces 114 on the outer ends of straps 110. The outer exo-skeletal member is further immobilized by the attachment 116 extending through the web space between the thumb and forefinger. The attachment 116 may be secured to member 106 by the rivet 117 and an equivalent rivet (not shown) on the opposite side, or may be adjustably secured using hook and loop type fasteners.

FIG. 11 shows a water hardenable splint blank 120 for providing splinting for the wrist and forearm 122. The blank 120 is provided with an attachment 124 for extending through the web space between the thumb and the forefinger. Preconnecting the attachment 124 to span the web allows for much easier and intuitive application of the support. The attachment or strap 124 may be riveted or sewn, for examples, to splint blank 120. The attachment 124 can be made from a thermoplastic, fabric, fiberglass, or combination of these materials, for examples. It is also possible for the attachment to be made from a material which has been treated to be water activatable, or alternatively may be unactivatable and remain soft and flexible for increased comfort during wear. This attachment may be permanently or removably attached and may have adjustment means, for example hook and loop type fasteners, to accommodate different sizes of anatomy. In addition, the tacking strap 126 having a patch of hook type material thereon, engages the hook receivable type patch 128 to quickly hold the blank 120 in place. The flaps 130 are wrapped around the thumb 132 to complete the splinting of the forearm and thumb, and may incorporate hook and loop type fasteners for quick securing during application. Functional securing in this select area is then achieved by the lamination of the layers of overlapping activatable flaps 130.

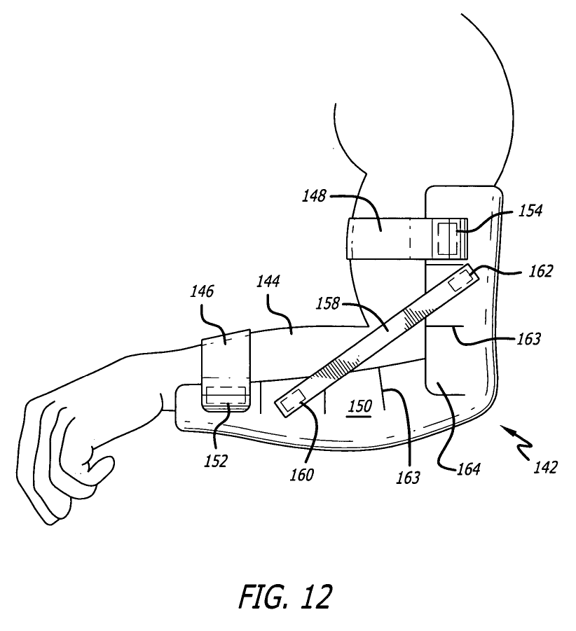

FIG. 12 shows a further embodiment illustrating the principles of the invention. In this embodiment, the splint 142 is mounted on the patient's arm 144. The tacking arrangements 146 and 148 are secured to the splint 150 at the rear of the splint. These securing points are not visible in FIG. 12 as they are on the opposite side of the arm. The tacking arrangements 146 and 148 have hook type patches 152 and 154 mounted on the ends thereof, which mate with a complementary hook receivable type material on the splint. This loop type material may be in the form of a patch on the splint 142, or the entire body 150 of splint 142 may have an outer layer of UBL or other hook receivable material thereon with which the hook type patches 152 and 154 engage and mate.

With the outer surface being covered with a UBL or other hook receivable material, an anti-flexing strap 158 may be utilized. The anti-flexing strap 158 may have hook type patches 160 and 162 at its ends, to mate with hook receivable type material on the body 150 of the splint. During application, the anti-flexing strap 158 aids in securing the arm and limiting the ability of the arm and splint to flex until it has hardened. After hardening, it will help reinforce the splint and keep the elbow at a ninety degree angle. If desired, the anti-flexion strap 158 may be permanently secured to splint 150 at one end thereof. As shown, the anti-flexion strap is used to help stabilize an elbow, however it may also be used to stabilize other necessary portions of the anatomy such as the ankle.

An additional feature shown in FIG. 12 is the ability to create slits 163 on both sides of the entire length of the splint. The slits allow for the splint to contour around complex curves of the anatomy without bunching and wrinkling. As shown in FIG. 12 when the splint is molded around the elbow, the splint material adjacent the slits overlap in the area 164 forming a lower profile splint with fewer wrinkles which might otherwise cause the user discomfort.

After the splint 142 is properly mounted in place, the entire splint assembly may be wrapped with flexible, resilient tape such as an ACE® type wrap, to functionally secure the splinting arrangements.

In regards to the materials used for fabricating the various layers of the above mentioned splinting assemblies, the tacking arrangements are preferably made from a nonwoven material. Nonwoven materials are typically very inexpensive and may be made to be hydrophobic which is very advantageous. The tacking materials may also be made of a knitted material, woven fabric, or foam. It is also beneficial if the tacking attachments are low profile, stretchable and tearable. The activatable layer can be composed of a double knit spacer material, multiple layers of a single knit material, foam laminate, nonwoven material, woven material, or any suitable material that will achieve sufficient strength upon hardening.

In closing, it is to be understood that the foregoing detailed description and the embodiments shown in the drawings are illustrative embodiments of the invention. Various alternatives and modifications may be made without departing from the spirit and scope of the invention. Thus, by way of example and not of limitation, in most of the embodiments shown herein, and specifically the embodiments of FIGS. 1-7 and 11, the functional securing may be accomplished using flexible elastic tape as indicated in FIG. 1, or straps as suggested in FIG. 10. Further, immobilization straps may be employed in all disclosed embodiments, in addition to the tacking arrangements as shown. Regarding the construction of the blanks, they may be of other forms than that shown in FIG. 2, and may include multiple layers of high strength material, for example, instead of a spacer material. Also, concerning hook receivable material, it may be in the form of patches of VELCRO® material, UBL material, or napped material, for examples. More generally the variations suggested by any of the drawings or related description are applicable to the other embodiments disclosed herein. Incidentally, instead of being water hardenable, the splints may include two materials with arrangements for combining the materials to initiate hardening. Accordingly, the present invention is not limiting the invention precisely as described in detail hereinabove and shown in the drawings.

While the specification describes particular embodiments of the present invention, those of ordinary skill can devise variations of the present invention without departing from the inventive concept

1. A method of applying splints or supports to an injured portion of the anatomy comprising the steps of:
forming a splint body, including a blank impregnated with hardenable material, said splint body having at least one primary laterally extending tacking extension;
activating said hardenable material;
holding said splint support to the anatomy by engaging said primary laterally extending tacking extension; and
functionally securing said splint or support in place with additional secondary immobilization arrangements;
whereby the primary tacking extensions permit convenient location and positioning adjustment of the splint or support during application, and the secondary securing arrangements provide functional support.

2. A method as defined in claim 1 wherein the splint or support is accurately positioned in the optimum position on the patient while the splint or support is held only by the primary tacking extensions.

3. A method as defined in claim 1 wherein said laterally extending securing extensions are formed as an integral part of said blank.

4. A method as defined in claim 1 wherein said splint or support is formed with an exterior surface of hook receivable material, and wherein each said tacking extension is provided with a hook type construction thereon which makes securing contact with said hook receivable material.

5. A method as defined in claim 1 wherein said blank is formed of double knit type spacer material.

6. An efficient splint or support comprising:
an orthopaedic blank impregnated with hardenable material;
primary tacking arrangements for holding said blank in place on the injured part of the patient's anatomy in a manner that still allows adjustment of said blank with respect to the anatomy; and
secondary holding arrangements for functionally securing the blank in place on the patient after the blank has been activated and properly mounted on the patient;
whereby the splint or support may be easily and properly mounted on the patient.

7. An efficient splint or support as defined in claim 6 wherein said blank is of generally longitudinal extent and has laterally extending extensions creating said tacking arrangements.

8. An efficient splint or support as defined in claim 6 wherein said blank has a covering formed of a hook receivable material and said primary tacking arrangements are provided with a hook type construction thereon.

9. An efficient splint or support as defined in claim 6 wherein said primary tacking arrangements are formed of hook and loop type material.

10. An efficient splint or support as defined in claim 6 wherein said blank is formed of double knit spacer type material.

11. An efficient splint or support as defined in claim 6 wherein said splint or support has an additional piece extending through the web space between the thumb and forefinger for assisting in the location of said splint on the forearm.

12. An efficient splint or support as defined in claim 11 wherein said tacking portion is integral to said splint or support.

13. An efficient splint or support as defined in claim 11 wherein said tacking portion is a separate and additional piece.

14. An efficient splint or support as defined in claim 6 wherein said secondary holding arrangements comprise an exo-skeletal structure overlying said blank.

15. An efficient splint or support as defined in claim 7 wherein said tacking arrangements are non-rectangular in shape.

16. An efficient splint or support as defined in claim 7 wherein at least one of said lateral extensions has an opening for receiving the thumb of a patient.

17. An efficient splint or support as defined in claim 6 wherein the primary tacking arrangement is made of a nonwoven material.

18. An efficient splint or support as defined in claim 6 wherein the primary tacking arrangements are removable and repositionable with respect to the splint or support.

19. An efficient splint or support as defined in claim 6 wherein the primary tacking arrangement is made from a stretchable material.

20. An efficient splint or support as defined in claim 6 wherein said orthopaedic blank has a padding layer on at least one side.

21. An efficient splint or support as defined in claim 6 wherein said secondary holding arrangements include a resilient, flexible, stretchable tape.

22. An efficient splint or support as defined in claim 6 wherein said blank has a non-rectangular shape and has laterally extending extensions creating said tacking arrangements.

23. An efficient splint or support as defined in claim 6 wherein said blank has slits along the length of the splint to reduce bulk after molding.

24. An efficient splint or support as defined in claim 6 wherein the splint or support assembly includes an anti-flexion strap for increased support.

25. An efficient splint or support comprising:
an orthopaedic blank impregnated with hardenable material;
primary tacking arrangements for holding said blank in place on the injured part of the patient's anatomy in a manner that still allows adjustment of said blank with respect to the anatomy; and
secondary holding arrangements comprising an exo-skeletal structure overlying said blank for functionally securing the blank in place on the patient after the blank has been activated and properly mounted on the patient;
whereby the splint or support may be easily and properly mounted on the patient.

26. An efficient splint or support as defined in claim 25 wherein said blank has a covering formed of hook receivable material and said primary tacking arrangements are provided with a hook type construction thereon.

27. An efficient splint or support as defined in claim 25 wherein said orthopaedic blank has a non-rectangular shape to fit a specific portion of the anatomy.

28. An efficient splint or support as defined in claim 25 wherein said blank is formed of double knit spacer type material.

29. An efficient splint or support as defined in claim 25 wherein said tacking arrangements are non-rectangular in shape.

30. A method of applying splints or supports to an injured portion of the anatomy comprising the steps of:
forming a splint body, including a blank impregnated with hardenable material, said splint body having at least one primary laterally extending tacking extension;
activating said hardenable material;
holding said splint support to the anatomy prior to hardening of the blank by engaging said primary laterally extending tacking extension;
adjusting the position and configuration of the blank while it is still malleable and is being loosely held by said tacking extension; and
maintaining the blank in its adjusted position while the blank hardens;
whereby the primary tacking extensions permit convenient location and adjustment of the splint or support during application of the splint or support and before the blank hardens.

31. A method as defined in claim 30 further comprising the step of functionally securing the splint or support in place with additional secondary immobilization arrangements.

32. A method as defined in claim 30 wherein said forming step includes forming said tacking extensions with hook and loop type materials.

33. A method as defined in claim 30 wherein said forming step includes forming said blank of double knit type spacer material.

34. A method as defined in claim 30 wherein said forming step includes forming said blank with a thumb hole.

35. A method as defined in claim 30 wherein said forming step includes forming said extensions with a non-rectangular configuration.

36. A method as defined in claim 30 wherein said forming step includes forming said tacking extension to be removably secured to said blank.

37. A method as defined in claim 30 wherein said forming step includes forming said blank with slits along the length of the splint or support.

38. A method as defined in claim 30 wherein said forming step includes forming the splint or support assembly with an anti-flexion strap for increased support.

39. An efficient splint or support method comprising the steps of:
forming a splint or support including an orthopaedic blank impregnated with water hardenable material;
holding said splint or support in place on the injured part of the patient's anatomy using primary tacking arrangements attached to said splint or support; and
functionally securing the splint or support in place on the patient after the blank has been activated and properly mounted on the patient, using secondary holding arrangements;
whereby the splint or support may be easily and effectively mounted on the patient.

40. An efficient splint or support comprising:
an elongate blank in roll form impregnated with hardenable material;
at least one layer of additional non-impregnated material on at least one side of said blank;
laterally extending primary tacking arrangements for holding said blank in place on the injured part of the patient's anatomy in a manner that still allows adjustment of said blank with respect to the anatomy; and
secondary holding arrangements for functionally securing the blank in place on the patient after the blank has been activated and properly mounted on the patient;
whereby the splint or support may be easily and properly mounted on the patient.

41. An efficient splint or support as defined in claim 40 wherein said additional layer is a padding material.

42. An efficient splint or support as defined in claim 40 wherein one side of said blank is covered with a padding material and the other with a hook receivable material and said primary tacking arrangements are provided with a hook type construction thereon.

43. An efficient splint or support as defined in claim 40 wherein said padding material is a double knit fabric.

44. An efficient splint or support as defined in claim 40 wherein said hook receivable material is UBL fabric.

45. An efficient splint or support as defined in claim 40 wherein said blank is formed of double knit spacer material.

46. An efficient splint or support as defined in claim 40 wherein said blank is formed of multiple layers of casting material.

47. An efficient splint or support as defined in claim 40 wherein said tacking arrangements are non-rectangular in shape.
